(19) United States
(12) Patent Application Publication
(10) Pub. No.: US 2021/0321634 A1
(43) Pub. Date: Oct. 21, 2021

(54) Lactobacillus Acidipiscis, Fermented Soymilk, and Preparation Method and Use Thereof

(71) Applicant: Hebei Agricultural University, Baoding (CN)

(72) Inventors: Xiuling Wang, Baoding (CN); Xia Guo, Baoding (CN); Xiumei Yu, Baoding (CN); Shijuan Dou, Baoding (CN); Mingyu Hou, Baoding (CN)

(21) Appl. No.: 17/307,956

(22) Filed: May 4, 2021

Related U.S. Application Data
(63) Continuation of application No. PCT/CN2020/095491, filed on Jun. 11, 2020.

(30) Foreign Application Priority Data
Jan. 13, 2020 (CN) CN202010031420.1

(51) Int. Cl. A23C 11/10; C12N 1/20

(52) U.S. Cl. CPC A23C 11/106; C12R 2001/225; C12N 1/205

(57) ABSTRACT

The present disclosure relates to the technical field of microorganisms and fermentation, and specifically discloses a Lactobacillus acidipiscis, a fermented soymilk, and a preparation method and use thereof. Lactobacillus acidipiscis HAU-FR7 is disclosed with a deposit number of CGMCC NO. 19253. The Lactobacillus acidipiscis HAU-FR7 is a facultative anaerobe that can reduce soy isoflavones, and the Lactobacillus acidipiscis HAU-FR7 can not only grow under aerobic conditions, but also convert daidzin and genistin distributed in the soymilk into DHD and DHG under aerobic conditions. Moreover, the Lactobacillus acidipiscis HAU-FR7 has stable conversion capacity, and solves the problem of shortage of facultative anaerobes in the research and development of soy functional foods. The DPPH radical-scavenging capacity of DHD and DHG is significantly higher than that of daidzein and genistein. Therefore, the discovery of the Lactobacillus acidipiscis HAU-FR7 will greatly promote the development and utilization of functional fermented soy products.

### CROSS-REFERENCE TO RELATED APPLICATIONS

This application is a continuation application of International Application No. PCT/CN2020/095491, filed on Jun. 11, 2020, which claims priority to Chinese Patent Application No.CN202010031420.1, filed on Jan. 13, 2020. The disclosures of the aforementioned applications are hereby incorporated herein by reference in their entireties.

### SEQUENCE LISTING

The application contains a Sequence Listing which has been submitted electronically in ASCII format and is hereby incorporated by reference in its entirety. Said ASCII copy, created on Jul. 12, 2021, is named 11300-010134-US0_ST25.txt and is 7 kilobytes in size.

### TECHNICAL FIELD

The present disclosure relates to a Lactobacillus acidipiscis, a fermented soymilk, and a preparation method and use thereof, and belongs to the field of microorganisms and fermentation.

### BACKGROUND

At present, use of probiotics by both domestic and overseas customers is very popular, however, the types of probiotics are still very limited. It is the lactic acid bacteria, which are of health care function, or extensively used in clinical. Researchers have isolated numerous lactic acid bacteria from different sources, and these isolated lactic acid bacteria have already been reported. There are also many reports on the use of lactic acid bacteria to ferment soymilk. In addition, some reports on soymilk fermentation by use of self-isolated lactic acid bacteria are also available.

As early as the 1980s, researchers tried to use lactic acid bacteria to ferment sterilized soymilk to prepare fermented soymilk. It is reported that in comparison with soymilk, both the anti-nutritional factors and the immunogenicity, in the fermented soymilk are decreased. Moreover, the content of amino acid, isoflavones, some antioxidant peptides and antihypertensive peptides is increased. Since fermented soymilk contains almost zero amount of cholesterol and does not contain lactose, it is especially suitable for people who are lactose intolerant or those who suffer from hypercholesterolemia. In addition, after being fermented by lactic acid bacteria, the proteins with high molecular weight in soymilk can be degraded into amino acids and polypeptides of different lengths. Studies have shown that lactic acid bacteria when being used to ferment soymilk can increase the total amino acid content. In addition, the soy peptides produced in the fermentation process by lactic acid bacteria are of antioxidant, and glucose, blood pressure and cholesterol lowering effects. Moreover, fermented soymilk, which is rich in soy isoflavones, is of the effects of preventing cardiovascular disease and cancer, anti-aging, especially to females, alleviating menopausal symptoms, preventing osteoporosis, and the like. Studies have shown that health care products with soy isoflavones as the main raw material have certain health care functions on human bones, blood, skin, sleep, immunity and other aspects.

Soy isoflavones are secondary metabolites formed during soybean growth. At present, there are 12 kinds of soy isoflavones that have been isolated and identified by chemical structures. Soy isoflavones are divided into two types: aglycons and glucosides, where most soy isoflavones exist as glucosides. Glucosides are mainly composed of daidzin and genistin. In soy isoflavones, the ratio of the aglycons, which are mainly composed of genistein and daidzein, is only 2% to 3%. After being absorbed, the glucosides can be hydrolyzed into aglycons by glycosidase. Aglycons are more lipid-soluble and can pass through the intestinal mucosal cells easily and exert physiological functions in blood. In addition, aglycons can also be converted by specific microbial strains in the intestine, where daidzein can be converted into dihydrodaidzein (DHD), tetrahydrodaidzein (THD), equol, O-desmethylangolensin (O-Dma), etc.; and genistein can be converted into dihydrogenistein (DHG), 2-(4-hydroxyphenyl) propionic acid (2-HPPA), 5-hydroxy-equol, etc. Many studies have shown that the microbial bioconversion products of soy isoflavones are of higher and broader biological activities than that of soy isoflavones.

At present, more than 30 bacterial strains with specific soy isoflavone bioconversion activities have been isolated and reported by researchers from different countries. On the bases of the different bioconversion activities, the soy isoflavone bioconverting bacteria can be divided into three groups: Group I, the bacterial strains with only hydrogenation reduction activity, can reduce daidzein and genistein to DHD and DHG under anaerobic conditions respectively; Group II, the bacterial strains with both hydrogenation reduction and ketone removal activity can convert daidzein and DHD to equol or genistein to 5-hydroxy-equol under anaerobic conditions respectively; and Group III, the bacterial strains with C-ring cleavage activity, can convert daidzein to O-desmethylangolensin (O-Dma), or convert genistein to 2-(4-hydroxyphenyl) propionic acid (2-HPPA) under anaerobic conditions.

Among more than 30 soy isoflavone bioconversion bacterial strains being reported, only 3 bacterial strains are facultative anaerobes. The first facultative anaerobic strain is Lactococcus sp. 20-92, which was isolated from human fecal samples by Japanese scholar Uchiyama et al. Strain Lactococcus sp. 20-92 can convert daidzein to equol, however, this bioconversion activity of the bacterial strain can work only under obligate anaerobic conditions. The second facultative anaerobic strain is Enterococcus hirae AUH-HM195, which was reported by the inventor's laboratory of the present application in 2009; Enterococcus hirae AUH-HM195 was isolated from Crossoptilon mantchuricum feces. Although the bacterial strain Enterococcus hirae AUH-HM195 can grow normally under aerobic conditions, it can only convert daidzein to O-Dma under anaerobic conditions. The third facultative anaerobic strain is Proteus mirabilis LH-52 which was isolated from the rat intestine and reported by Xiao Meitian's research group in Huaqiao University of China in 2012. However, it was reported that strain Proteus mirabilis LH-52 showed unstable bioconversion activity when being cultured under aerobic conditions.

The reason why the fermented soymilk has stronger antioxidant activity in comparison with soymilk, is mainly due to the two aspects: firstly, the amount of aglycons is significantly increased, and secondly, the soy peptides and some metabolites produced in the fermentation process by the lactic acid bacteria are of stronger antioxidant activity.

However, no facultative anaerobes, which can reduce daidzein and genistein under aerobic conditions, have been reported either by domestic or by overseas scholars.

### SUMMARY

These and other problems are generally solved or circumvented, and technical advantages are generally achieved, by embodiments of the present disclosure which provides a Lactobacillus acidipiscis, a fermented soymilk, and a preparation method and use thereof.

### Technical Problems

In view of shortage of facultative anaerobes with soy isoflavone reducing activity for research and development of soy functional foods, the present disclosure provides a Lactobacillus acidipiscis, a fermented soymilk, and a preparation method and use thereof.

### Technical Solutions

Lactobacillus acidipiscis HAU-FR7, with a collection number of CGMCC NO. 19253, is disclosed.

A lactic acid bacterium was isolated from traditional Chinese fermented soy product designated stinky tofu. After being identified, we named the isolated bacterium as Lactobacillus acidipiscis HAU-FR7, and deposited in the China General Microbiological Culture Collection Center, referred to as CGMCC on Dec. 27, 2019, with a collection number of CGMCC NO. 19253, and the preservation address is Institute of Microbiology, Chinese Academy of Sciences, No. 3, Courtyard 1, Beichen West Road, Chaoyang District, Beijing.

In the present disclosure, the pathway that Lactobacillus acidipiscis HAU-FR7 converts daidzin to DHD and genistin to DHG is shown in FIG. 1.

The taxonomic characters of the Lactobacillus acidipiscis HAU-FR7 in the present disclosure are as follows.

The Lactobacillus acidipiscis HAU-FR7 is Gram positive and catalase negative, has round colonies with neat edges, white colonies on MRS agar medium and the diameter of the colony is from 0.5 mm to 1.5 mm. When being grown in MRS liquid medium, the bacterial cells are short rod-shaped and arranged individually; in the growth process, the MRS liquid medium is turbid; and in the process of the stable period, the bacterial cells all sink to the bottom.

The Lactobacillus acidipiscis HAU-FR7 is oxidase negative, production of ammonia from arginine negative, urease negative, H2S negative, V.P. test negative, gelatin liquefaction negative, and starch hydrolysis weakly positive, has strong ability to use glucose, fructose and inulin, has weak ability to use lactose, maltose and xylitol, and cannot use sucrose orsorbitol.

The present disclosure further provides a use of the Lactobacillus acidipiscis HAU-FR7 in fermentation of soymilk.

The Lactobacillus acidipiscis HAU-FR7 provided by the present disclosure can not only grow normally under aerobic conditions, but also has high bioconversion activity to both daidzin and genistin in soymilk under aerobic conditions, and can further efficiently bioconvert daidzin to DHD and genistin to DHG. The 1,1-diphenyl-2-picrylhyclrazyl (DPPH) free radical scavenging activity of DHD and DHG is significantly higher than that of daidzein and genistein, and the cardiovascular protective activity of DHD is also significantly higher than that of daidzein. Therefore, the Lactobacillus acidipiscis HAU-FR7 shows good application value in the field of preparing functional fermented soymilk.

The present disclosure further provides a fermented soymilk, prepared by using the Lactobacillus acidipiscis HAU-FR7.

The present disclosure further provides a method for fermenting soymilk by using the Lactobacillus acidipiscis HAU-FR7, including the steps of: inoculating a cultural broth of Lactobacillus acidipiscis HAU-FR7 in a logarithmic growth phase (the OD (Optical density) value at 600 nm of the cultural broth is in the range of 1.0 to 1.5, or the OD value at 600 nm of the cultural broth may be 1.0 or more) into a soymilk sterilized at 121° C. for 15 min in advance, and fermenting the soymilk at 25° C.-42° C. for 36 hours (h)-50 h to obtain the fermented soymilk.

In one embodiment, the OD (Optical density) value at 600 nm of the cultural broth of the Lactobacillus acidipiscis HAU-FR7 in the logarithmic growth phase is 1.0 or more.

In one embodiment, the inoculation amount of the cultural broth of the Lactobacillus acidipiscis HAU-FR7 is 8%-12% of the volume of the soymilk.

In one embodiment, the soymilk is prepared by mixing and pulverizing soaked soybeans and water at a weight-to-volume ratio of 1:6, where a unit of weight is gram, and a unit of volume is milliliter.

In one embodiment, a preparation method of the cultural broth of the Lactobacillus acidipiscis HAU-FR7 includes the steps of: inoculating an activated bacterial solution of the Lactobacillus acidipiscis HAU-FR7 into fresh MRS liquid medium at an inoculation amount of 4%-6% (v/v), and culturing in the MRS liquid medium at 35° C.-40° C. for 20 h-25 h to obtain the cultural broth of the Lactobacillus acidipiscis HAU-FR7.

The activated bacterial solution of the Lactobacillus acidipiscis HAU-FR7 is obtained by gradually melting a glycerol cryopreservation tube of Lactobacillus acidipiscis HAU-FR7 in an ice water mixture, followed by inoculating the melted Lactobacillus acidipiscis HAU-FR7 into a test tube filled with fresh MRS liquid medium at an inoculation amount of 10%-15% (v/v), and culturing the MRS liquid medium at 37° C. When The OD₆₀₀ value of the cultural broth of Lactobacillus acidipiscis HAU-FR7 is in the range of 1.0 to 1.5, or the OD value at 600 nm of the cultural broth may be 1.0 or more, the cultural broth was inoculated into the test tube filled with fresh MRS liquid medium at the inoculation amount of 10%-15% (v/v). When the OD600 value of the cultural broth is in the range of 1.0 to 1.5, or the OD value at 600 nm of the cultural broth may be 1.0 or more, the activated bacterial solution was prepared.

The present disclosure further provides a use of the fermented soymilk in preparation of healthy foods for resisting oxidation and aging, enhancing immunity, and lowering blood pressure or blood lipid.

### ADVANTAGEOUS EFFECTS OF THE DISCLOSURE

The Lactobacillus acidipiscis HAU-FR7 provided by the present disclosure is a facultative anaerobe that can convert soy isoflavones. The Lactobacillus acidipiscis HAU-FR7 can not only grow under aerobic conditions, but also convert daidzin distributed in the soymilk into daidzein, and genistin distributed in the soymilk into genistein, under aerobic conditions; importantly, the Lactobacillus acidipiscis HAU-FR7 can further reduce daidzein and genistein to DHD and DHG respectively and efficiently. The Lactobacillus acidipiscis HAU-FR7 has stable conversion capability, and solves the problem of shortage of facultative anaerobes for converting soy isoflavones in the research and development of soy functional foods. The DPPH free radical scavenging activity of DHD and DHG at a certain concentration is significantly higher than that of daidzein and genistein respectively. Therefore, the discovery of the Lactobacillus acidipiscis HAU-FR7 will greatly promote the development and utilization of functional fermented soy products.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

FIG. 1 is a schematic diagram of an embodiment of pathway of the Lactobacillus acidipiscis HAU-FR7 of the present disclosure to convert daidzin to DHD, and genistin to DHG;

FIG. 2 shows an example of high performance liquid chromatogram of authentic genistein, authentic daidzein and a crude soy isoflavone extract under (i) Extraction of isoflavones from soybeans in Embodiment 1, where a. the authentic genistein, b. the authentic daidzein, and c. crude soy isoflavone extract;

FIG. 3 is an ultraviolet (UV) absorption spectrum of peak 1 in the high performance liquid chromatogram of the crude soy isoflavone extract in FIG. 2;

FIG. 4 is an ultraviolet absorption spectrum of peak 2 in the high performance liquid chromatogram of the crude soy isoflavone extract in FIG. 2;

FIG. 5 shows an example of high performance liquid chromatogram of authentic dihydrogenistein, authentic dihydrodaidzein and a crude soy isoflavone extract converted by a bacterial strain in an MRS liquid medium under (iii) Isolation and screening of bacterial strains with conversion function in Embodiment 1 of the present disclosure, where a. the authentic dihydrogenistein, b. the authentic dihydrodaidzein, and c. the crude soy isoflavone extract fermented by Lactobacillus acidipiscis HAU-FR7 in the MRS liquid medium;

FIG. 6 is an ultraviolet absorption spectrum of an unknown peak 1 in the high performance liquid chromatogram of the crude soy isoflavone extract converted by the bacterial strain in the MRS liquid medium in FIG. 5;

FIG. 7 is an ultraviolet absorption spectrum of an unknown peak 2 in the high performance liquid chromatogram of the crude soy isoflavone extract converted by the bacterial strain in the MRS liquid medium in FIG. 5;

FIG. 8 shows an example of high performance liquid chromatogram of fermented soymilk by Lactobacillus acidipiscis HAU-FR7 and high performance liquid chromatogram of a mixed solution containing authentic dihydrogenistein, authentic dihydrodaidzein, authentic genistein and authentic daidzein under (2) Changes in soy isoflavones during fermentation in Embodiment 5 of the present disclosure, where a. the authentic dihydrogenistein, the authentic dihydrodaidzein, the authentic genistein and the authentic daidzein; and b. bacterial fermentation broth of soymilk;

FIG. 9 is a diagram showing comparison in DPPH free radical scavenging activity of soymilk before and after being fermented by Lactobacillus acidipiscis HAU-FR7 at different detection concentrations; and

FIG. 10 is a diagram showing comparison in DPPH free radical scavenging activity of soymilk of a certain concentration before and after being fermented by Lactobacillus acidipiscis HAU-FR7 within different reaction time periods.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION OF ILLUSTRATIVE EMBODIMENTS

1. Screening of Lactobacillus acidipiscis HAU-FR7

(1) Isolation of Lactobacillus acidipiscis HAU-FR7 from commercially available stinky tofu.

(i) Extraction of isoflavones from soybeans

The isoflavones were crudely extracted from soybeans with ethyl acetate. After the ethyl acetate was evaporated to dryness by a rotary evaporator, chromatographic grade methanol was added. The prepared crude soy isoflavone extract was used as the substrate (the sum of the concentration of daidzin and that of genistin is in the range of 0.3 mmol/L to 0.5 mmol/L), which was detected by high performance liquid chromatography (HPLC).

High performance liquid chromatography system: 1525 type double pump and 2487 UV detector of American Waters company; Chromatographic column: Elite C₁₈ analytical column (5 μm, 250 mm×4.6 mm).

Mobile phase: The mobile phase comprises 10% (v/v) acetonitrile solution in water (solution A) buffered with 0.1% (v/v) acetic acid, and 90% (v/v) acetonitrile solution in water (solution B) buffered with 0.1% (v/v) acetic acid, gradient elution is performed, and the elution procedures are as follows:

0-8 min, 70% solution A, 30% solution B;

8-15 min, 70%→50% solution A, 30%→50% solution B;

15-20 min, 50%→70% solution A, 50%→30% solution B;

Detection wavelength: 270 nm;

Flow rate: 1.0 mL/min;

Injection volume: 20 μL.

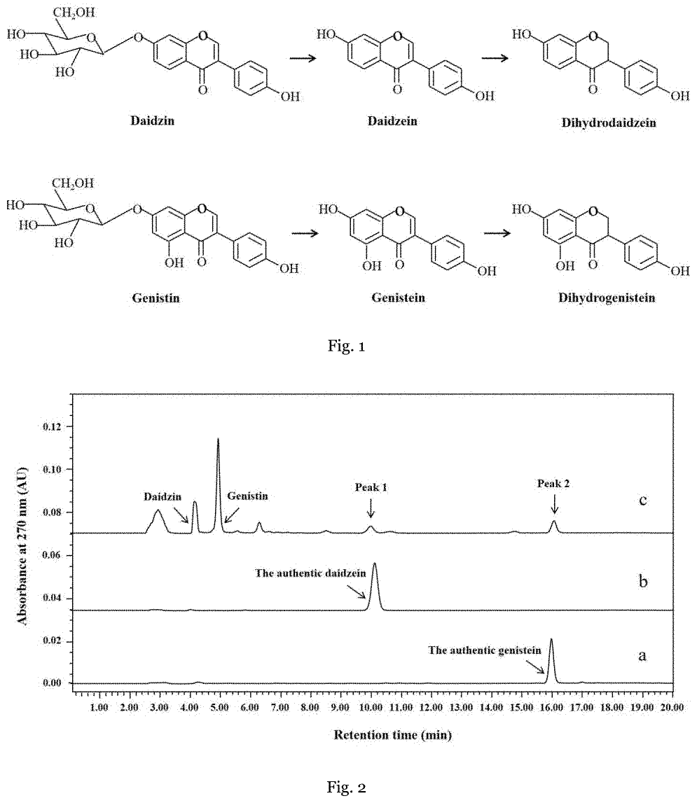

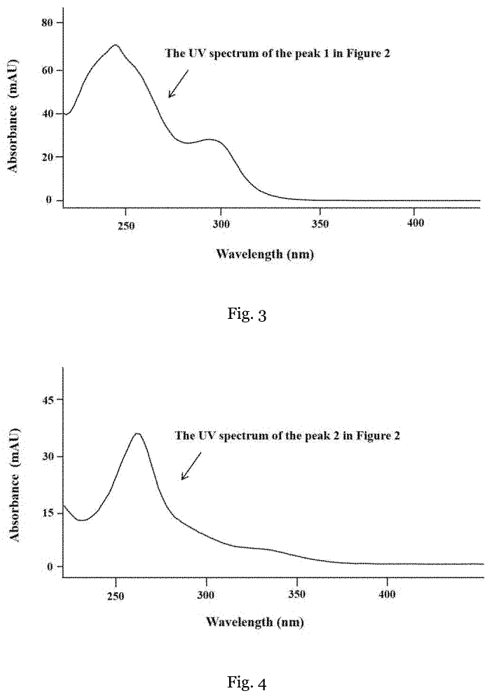

The detection results are shown in FIG. 2. Due to the high hydrophilicity of glucosides, when the water content in the mobile phase is much higher than that of the acetonitrile, glucosides peaks usually come out quickly in the HPLC elution profile. In FIG. 2, the substance peak with a retention time of about 4 min is daidzin, and the substance peak with a retention time of 5 min is genistin in the HPLC elution profile. In addition, according to the retention time of the authentic daidzein and the authentic genistein in FIG. 2 as well as the UV absorption spectra of the peak 1 and the peak 2 of the crude soy isoflavone extract in the HPLC elution profile (FIG. 3 and FIG. 4), in the crude soy isoflavone extract, the substance peak 1 with a retention time of about 10 min was identified as daidzein, and the substance peak 2 with a retention time of about 16 min was identified as genistein.

(ii) Gradient Dilution

The brine and the fermented bean curd in the commercially available stinky tofu were mixed evenly to obtain a mixed solution. The mixed solution was diluted serially from 10⁴ to 10⁻⁵ in a MRS liquid medium. Subsequently, 50 μL of each of the serially diluted solution was spread on MRS agar media in duplicate, being cultured in an anaerobic chamber and an ordinary biochemical incubator at 37° C. for 2-3 days, respectively. The colony morphology was observed and recorded.

(iii) Isolation and Screening of Bacterial Strains with Isoflavone Bioconvering Activities

In step (ii), a variety of single colonies with different morphologies were obtained. Each single colony was cultured in an MRS liquid medium. A crude soy isoflavone extract (the sum of the concentration of daidzin and that of genistin is in the range of 0.3 mmol/L to 0.5 mmol/L) was added to the MRS liquid medium as a substrate. The MRS liquid medium with soy isoflavone substrate in it was co-cultured for 3 days at 37° C. in an ordinary biochemical incubator and then a cultural broth was obtained. Subsequently, 200 μL of the cultural broth was taken out and extracted with 1000 μL of ethyl acetate. The extract was filtered with organic filter membrane, the pore size of which is 0.45 μm. The filtered extract was evaporated to dryness with a rotary evaporator, then a certain amount of 100% chromatographic grade methanol was added to obtain a test solution. The concentration changes of daidzin, genistin, daidzein and genistein in the test solution as well as the appearance of new peaks were detected using high performance liquid chromatography.

It was found that when one colony was co-cultured with the crude soy isoflavone extract, the amount of the glucosides was decreased, where the concentration of daidzin was decreased from 0.184 mmol/L to 0.102 mmol/L, and that of the genistin decreased from 0.222 mmol/L to 0.123 mmol/L. The results indicated that the bacterial strain can produce glycosidase, and the bacterial strain was named HAU-FR7.

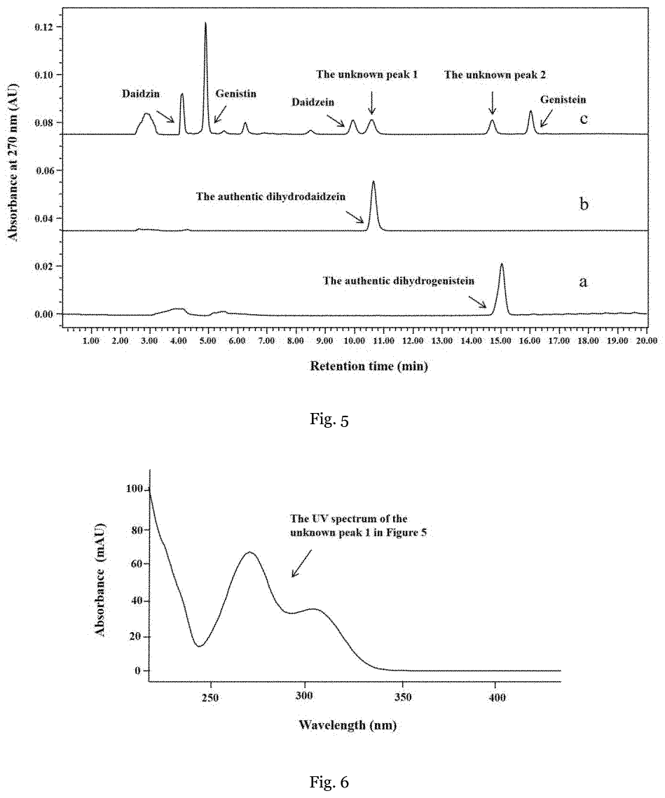

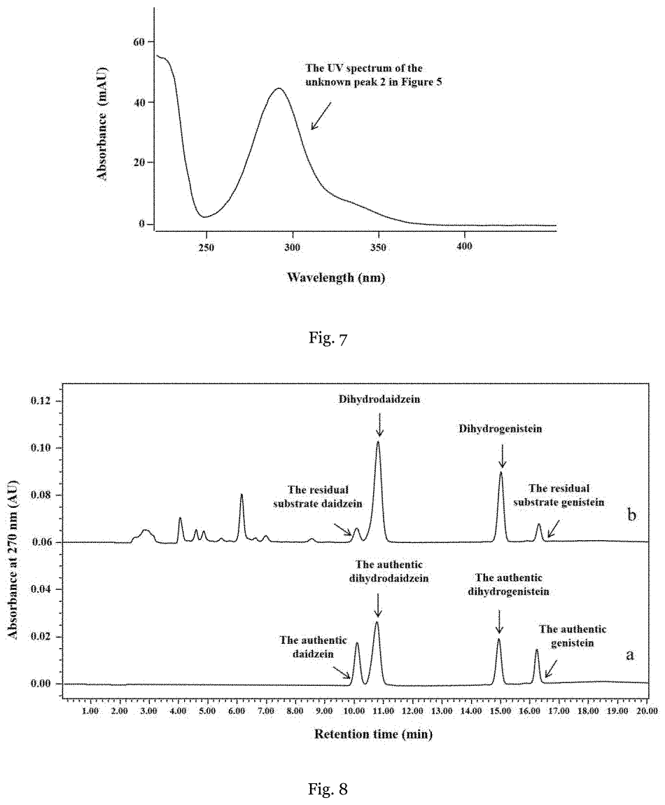

In addition, as shown in FIG. 5, the high performance liquid chromatography also detected two new peaks, the retention time which was of 10.6 min and 14.7 min, respectively. We named the peak appeared at 10.6 min unknown peak 1, and that appeared at 14.7 min unknown peak 2. According to the high performance liquid chromatography retention time of the authentic DHD and the authentic DHG, as well as the characteristics of the UV spectra of the unknown peak 1 and the unknown peak 2 (FIG. 6 and FIG. 7), the unknown peak 1 was preliminarily identified as DHD, the hydrogenation reduction product of daidzein, and the unknown peak 2 was preliminarily identified as DHG, the hydrogenation reduction product of genistein.

(iv) Mass Spectrometric Analysis

In order to further determine the structure of the unknown products, the unknown products were separated and purified by a high performance liquid chromatograph and subjected to cation mass spectrometric analysis. The result showed that the mass spectrum of the unknown peak 1 is: ESI(+): m/z 257 ([M+H]⁺); MS/MS (rel. int. %): m/z 137(67), 120(57), 91(31), indicating that the molecular weight of the unknown peak 1 is 256, which is exactly the same as that of DHD. Therefore, according to the retention time of the high performance liquid chromatograph, UV absorption spectrum and the detected mass spectrum, the unknown peak 1 produced from daidzein by the bacterial strain HAU-FR7 was accurately identified as DHD.

Similarly, the purified unknown peak 2 was subjected to mass spectrometry, and the result showed by mass spectrum is: ESI(+): m/z 273 ([M+H]⁺); MS/MS (rel. int. %): m/z153(82), 120(36), 91(25), 65(7), indicating that the molecular weight of the unknown peak 2 is 272, which coincides with that of DHG. Therefore, according to the retention time of the high performance liquid chromatograph, UV absorption spectrum and the detected mass spectrum, the unknown peak 2 produced from genistein by the bacterial strain HAU-FR7 was accurately identified as DHG.

(2) Purification, Strain Identification and Preservation of Lactobacillus acidipiscis HAU-FR7

(i) Strain Purification and Culture Preservation

The isolated colonies with soy isoflavone bioconverting activity were streaked and cultured on an MRS agar medium. After single colonies grew, the grown single colonies were re-streaked. The streaking process was repeated at least 3 times to ensure that the morphologies of the grown single colonies were exactly the same. The purified single colonies were inoculated into 4 mL of MRS liquid medium and cultured for 24 h. Subsequently, 200 μL of the cultural broth was taken out and added to a cryopreservation tube containing 200 μL of 50% (v/v) glycerol aqueous solution sterilized in advance. A solution was mixed well and stored in an ultra-low temperature refrigerator at −80° C. The stored bacterial strains were rejuvenated and the conversion activity was determined regularly.

(ii) Strain Identification

Using the total DNA of the bacterial strain HAU-FR7 as the template, and the universal primer 27F/1492R (27F: 5′-AGAGTTTGATCCTGGCTCAG-3′; 1492R: 5′-GGTTACCTTGTTACGACTT-3′) as the primer, the 16S rDNA sequence was amplified by Polymerase Chain Reaction (PCR). The PCR amplification product was sent to Shanghai Bioengineering Co., Ltd. to perform DNA sequencing. The 16S rDNA sequence of strain HAU-FR7 was subjected to BLAST alignment with other bacterial strains in the GenBank database for similarity analysis. Through BLAST alignment, the 16S rDNA sequence of the bacterial strain HAU-FR7 has the highest similarity with that of Lactobacillus acidipiscis strain NBRC 102163 (NR 112693.1), the similarity of which is 99.79%, and the similarity with that of Lactobacillus pobuzihii strain E100301 (NR 112694.1) is 98.32%. Combined with the physiological and biochemical characteristics of the bacterial strain HAU-FR7, the functional lactic acid bacteria HAU-FR7 isolated from the Chinese traditional stinky tofu was preliminarily identified as Lactobacillus acidipiscis, namely Lactobacillus acidipiscis HAU-FR7.

Preparation of seed fermentation broth of the Lactobacillus acidipiscis HAU-FR7 is as follows: the glycerol cryopreservation tube of the Lactobacillus acidipiscis HAU-FR7 was melted gradually in an ice water mixture, followed by being inoculated into a test tube containing fresh MRS liquid medium and cultured at 37° C. for 48 h, the inoculation amount of which was 10%-15% (v/v); and then, the Lactobacillus acidipiscis HAU-FR7 in the test tube was transferred into a fresh MRS liquid medium at an inoculation amount of 5% (v/v), and cultured for 12 h-18 h to be used as the seed fermentation broth.

2. Evaluation of Safety and Probiotic Characteristics of Lactobacillus acidipiscis HAU-FR7

(1) Analysis of the Ability of Lactobacillus acidipiscis HAU-FR7 to Produce Biogenic Amine

The Lactobacillus acidipiscis HAU-FR7 frozen in the glycerol cryopreservation tube was melted and the seed fermentation broth was prepared. The prepared seed fermentation broth was inoculated in MRS liquid medium containing 0.1% (w/v) lysine (or 0.1% (w/v) tyrosine, or 0.1% (w/v) histidine) and 0.005% (w/v) pyridoxal-5-phosphate to induce the production of cadaverine, tyramine and histamine, respectively. After 5 passages, 5 μL of the bacterial cultural broth was dropped onto agar detection media containing lysine, tyrosine or histidine, respectively. After the agar detection media being cultured in a 37° C. incubator for 48 h, the color change of the media near the colony was observed. On the one hand, if the color of the agar media near the colony becomes yellow, it stands for a negative result, indicating that no biogenic amine is produced or the amount of biogenic amine produced is too low to change the color of the agar medium. On the other hand, if the color of the agar medium near the colony becomes red, it stands for a positive result, indicating that biogenic amine is produced. According to the test results, the color of all the media near the colony is yellow, which indicated that the bacterial strain HAU-FR7 does not produce biogenic amine, such as cadaverine, tyramine or histamine.

(2) Antibiotic Tolerance Test of Lactobacillus acidipiscis HAU-FR7

The sensitivity of the bacterial strain HAU-FR7 to antibiotics, including cefoperazone, gentamicin, erythromycin, ampicillin, meropenem, tetracycline, vancomycin, levofloxacin, trimethoprim, rifampicin and penicillin G, was determined by the drug disc diffusion method. The cultural broth of the bacterial strain HAU-FR7 in MRS medium was spread on an MRS agar medium. After about 15 min, a presterilized susceptibility paper with a diameter of 6 mm containing different antibiotics was pasted on the MRS agar medium respectively by using a sterile tweezers, followed by being incubated at 37° C. for 24 h. Subsequently, the diameter of an inhibition zone was observed and measured.

On the basis of the sensitivity test of the 11 kinds of antibiotics, it is found that Lactobacillus acidipiscis HAU-FR7 is sensitive to all of the tested antibiotics, indicating that Lactobacillus acidipiscis HAU-FR7 is intolerant to the 11 kinds of tested antibiotics.

(3) Determination of Tolerance of Lactobacillus acidipiscis HAU-FR7 to bovine bile salt

The bacterial strain HAU-FR7 was cultured to the logarithmic growth phase. 5 portions of the cultural broth, containing 1 mL of each, were taken out and centrifuged at 8000 r/min for 10 min. The supernatant was discarded and 5 portions of bacterial cell pellet were obtained. Then 1 mL of solution containing 0.1% (w/v) bovine bile salt was added to the first portion of the bacterial cell pellet, 1 mL of solution containing 0.2% (w/v) bovine bile salt was added to the second portion of the bacterial cell pellet, 1 mL of solution containing 0.3% (w/v) bovine bile salt was added to the third portion of the bacterial cell pellet, and 1 mL of solution containing 0.5% (w/v) bovine bile salt was added to the fourth portion of the bacterial cell pellet. 1 mL of PBS buffer with a pH of 6.5 was added to the fifth portion of the bacterial cell pellet as a contrast. All the 5 portions of solutions mentioned previously were mixed evenly and respectively followed by being cultured in a 37° C. incubator for 3 h. 100 μL of each culture solution was taken out to be subjected to gradient dilution. The dilutions were spread on MRS agar media contained in petri dishes, and the petri dishes with different spreading dilutions were placed in a 37° C. incubator and cultured overnight. Colonies were counted to calculate the survival rate of the bacterial strains under different bovine bile salt concentrations.

The test results showed that the survival rate of the bacterial strain HAU-FR7 after being treated under bile salt concentrations of 0.1% (w/v), 0.2% (w/v), 0.3% (w/v) and 0.5% (w/v) for 3 h was 86.29%, 86.00%, 36.39% and 1.25%, respectively, indicating that the bacterial strain HAU-FR7 has a certain tolerant capability to bovine bile salt.

(4) Determination of Tolerance of Lactobacillus acidipiscis HAU-FR7 to Artificial Gastrointestinal Juice

3 portions of the cultural broth containing solution 1 mL of each, cultured to the logarithmic growth phase, were taken out and centrifuged at 8000 r/min for 10 min. The supernatant was discarded and 3 portions of bacterial cell pellet were obtained. Then 1 mL of the artificial gastric juice was added to the first portion of the bacterial cell pellet, and 1 mL of the artificial intestinal juice was added to the second portion of the bacterial cell pellet, 1 mL of PBS buffer with a pH of 6.5 was added to the third portion of the bacterial cell pellet as a contrast. After being mixed evenly, the 3 portions of solutions were placed in a 37° C. incubator and cultured for 3.0 h; 100 μL of each of the 3 portions of solutions was taken out to make serial dilution after being incubated for 0 h,0.5 h and 3.0 h, respectively. The dilutions were spread on MRS agar media contained in petri dishes, and the petri dishes with different spreading dilutions were placed in a 37° C. incubator and cultured overnight. Colonies were counted to calculate the survival rate of the strains under different reaction time in the artificial gastrointestinal juice.

The results showed that the survival rate of the bacterial strain HAU-FR7 was similar to that of the contrast when treated for 0.5 h and 3.0 h in the artificial intestinal juice, indicating that the bacterial strain HAU-FR7 was hardly affected by trypsin or high pH in the simulated artificial intestinal juice. However, when treated with the simulated artificial gastric juice, about half of the bacterial cells could not survive after only 0.5 h of incubation; after 3 h of treatment, the number of bacteria survived was close to zero, indicating that the bacterial strain HAU-FR7 has a low tolerance to the artificial gastric juice.

3. Preparation Method of Fresh Soymilk:

Plump soybean seeds with no obvious moth-eaten damage were selected. After being washed, the soybeans were soaked in water containing 0.5% NaHCO₃ (w/v) at room temperature for 14 h until the two cotyledons of the soybeans could be easily separated by hand. After the soaked soybeans being washed for 2 or 3 times, drinking water was added to till the soybean-water ratio becomes 1:6 (w/v), and a soymilk machine was used to make fresh soymilk. After the soymilk was made, containers of a suitable size were selected to dispense the soymilk evenly. Each container was dispensed with 15 mL of soymilk. After sealing, the containers were sterilized using a vertical pressure steam sterilizer at 121° C. for 15 min, and cooled to room temperature to obtain the sterilized fresh soymilk.

4. Method for Fermenting Soymilk with Lactobacillus acidipiscis HAU-FR7

When the Lactobacillus acidipiscis HAU-FR7 seed fermentation broth in the MRS liquid medium was cultured to the logarithmic growth phase, the seed fermentation broth was inoculated into fresh soymilk sterilized in advance at an inoculation amount of 5% of the volume of the soymilk, and fermented at 37° C. for 48 h.

5. Method for Detecting the Quality of Fermented Soymilk:

(1) Evaluation of the Acid Production Capacity of Lactobacillus acidipiscis HAU-FR7

The production process for the fermented soymilk is the same as mentioned previously. After being inoculated, samples were taken out at 0 h, 3 h, 6 h, 9 h, 12 h, 18 h, 24 h, 36 h, and 48 h of incubation to determine the pH value of the soymilk during different fermentation period. The results showed that the pH of the initially fermented soymilk was about 6.5, and the pH of the fermented soymilk decreased gradually with the time. When the fermentation time was about 18 h, the pH of the fermented soymilk dropped down to 4.8, which was the lowest level, and then the pH of the fermented soymilk kept being stabilized.

(2) Changes of Soy Isoflavones in Fermentation Process

The production process for the fermented soymilk is the same as mentioned previously. The inoculation amount was 5% of the volume of soymilk. The soymilk was fermented at 37° C. for 48 h. And 2 mL of the fermented soymilk was taken out after being incubated for different time periods. 10 mL of ethyl acetate was added and shaken for extraction. The extract was centrifuged at 8000 r/min for 10 min. The supernatant was taken out and filtered with organic filter membrane, the pore size of which is 0.45 μm. 400 μL of the filtered extract was taken out and evaporated to dryness by using a rotary evaporator. 80 μL of chromatography grade methanol was added. The concentration changes of the soy isoflavones and the products in the soymilk fermented at different time periods were detected by high performance liquid chromatography. The mixed solutions of the authentic dihydrogenistein, the authentic dihydrodaidzein, the authentic genistein and the authentic daidzein were taken as the contrast, where the concentration of dihydrogenistein and dihydrodaidzein was 0.1 mmol/L respectively, and that of genistein and daidzein was 0.04 mmol/L respectively.

From FIG. 8, after the soymilk was fermented by the bacterial strain HAU-FR7 for 48h, more than 90% of the daidzin in the soymilk can be converted into daidzein, and further reduced to dihydrodaidzein (DHD); and similarly, more than 90% of the genistin is converted to genistein and further reduced to dihydrogenistein (DHG).

On the basis of the same fermentation process, when the soymilk inoculated with the bacterial strain HAU-FR7 was fermented at 25° C.-42° C. for 36 h-50 h, the conversion efficiency was similar to that mentioned previously.

(3) Effect of Different Carbohydrates on Conversion Effect of Soy Isoflavones in Soymilk

The soymilk was prepared as mentioned previously, however, during soymilk dispensing process, 1%, 4% and 8% of sucrose, glucose or maltose was added to the soymilk before sterilization. The soymilk with different concentration or different kinds of carbohydrates was sterilized at 121° C. for 15 min, and then inoculated with Lactobacillus acidipiscis HAU-FR7after cooling. Soymilk without any sugars was used as a contrast. The inoculation concentration was 5% of the volume of the soymilk. After 36 h of fermentation at 37° C., the soymilk was extracted with ethyl acetate before and after fermentation, and the soy isoflavone bioconversion capacity by the bacterial strain HAU-FR7 was detected by high performance liquid chromatography.

(i) Effect of Glucose on the Conversion Capacity of Lactobacillus acidipiscis HAU-FR7

In the present disclosure, 1% (w/v), 4% (w/v) and 8% (w/v) of glucose were added to soymilk respectively. After fermentation, the changes in concentration of soy isoflavone glycosides and aglycones in soymilk were detected by high performance liquid chromatography. The results showed that the addition of glucose at different concentrations significantly reduced the conversion capacity of the bacterial strain HAU-FR7 (P<0.01). The results showed that the addition of glucose to the soymilk seriously influenced the soy isoflavone conversion capacity of the bacterial strain HAU-FR7 in the soymilk fermentation process.

(ii) Effect of Sucrose on the Conversion Capacity of Lactobacillus acidipiscis HAU-FR7

In addition to glucose, the present disclosure further explored the effect of sucrose added in the soymilk on the conversion capacity of the bacterial strain HAU-FR7.It was found that the addition of sucrose had significant influence neither on the hydrolyzation capacity of soy isoflavone aglycosides to form daidzein and genistein nor the reduction conversion capacity of isoflavone aglycones daidzein and genistein to form dihydrodaidzein and dihydrogenistein respectively.

(iii) Effect of Maltose on Conversion Capacity of Lactobacillus acidipiscis HAU-FR7

In the present disclosure, 1% (w/v), 4% (w/v) and 8% (w/v) of maltose were added to soymilk respectively. After fermentation, the conversion of soy isoflavone glycosides and aglycones in soymilk was detected by high performance liquid chromatography. The results showed that the addition of maltose significantly decreased the reduction capacity of the bacterial strain HAU-FR7 (P<0.05), however, the influence was relatively weaker in comparison to that of glucose.

6. Detection of DPPH Radical-Scavenging Capacity of the Fermented Soymilk by Lactobacillus acidipiscis HAU-FR7

(1) Effect of Different Concentrations of Fermented Soymilk on DPPH Radical-Scavenging Tatio

800 μL of 0.1 mmol/L DPPH-ethanol solution were taken and respectively mixed with 0.00 μL, 6.25 μL, 12.50 μL, 25.00 μL, 50.00 μL, 100.00 μL and 200.00 μL of the fermented soymilk mentioned previously. The mixed solution was adjusted the volume to 1 mL with distilled water. After being shook adequately, the reaction was carried out in the dark at 25° C. for 30 min. Then the sample was centrifuged at 8000 r/min for 5 min, and the supernatant was taken out to measure the absorbance at 517 nm. The DPPH radical-scavenging ratio was calculated by the following formula:

Scavenging ratio of DPPH free radicals =(Ao-Ai)/Aox100%, where Ao is the blank absorbance value; and Al is the sample absorbance value.

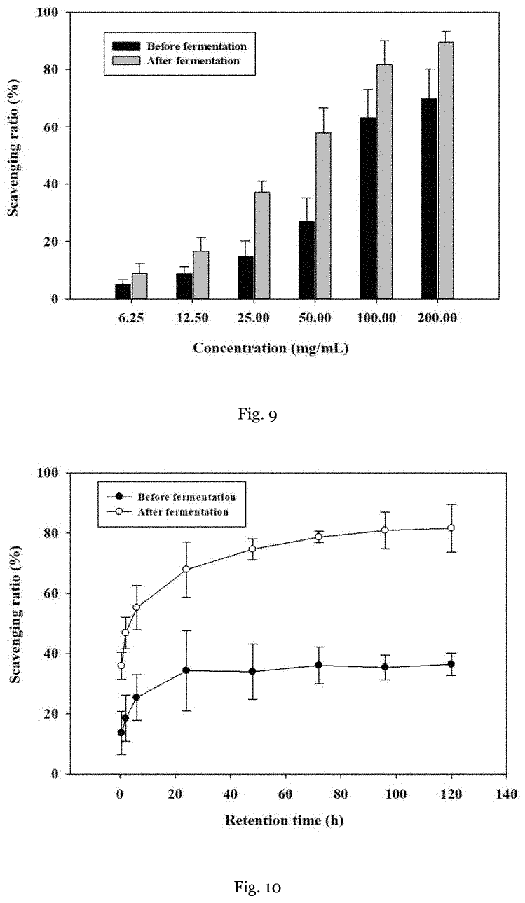

In the DPPH free radical scavenging test in the present disclosure, when the concentration of the unfermented soymilk and that of the fermented soymilk was 6.25 mg/mL, the DPPH free radical scavenging ratio of the unfermented soymilk and that of the fermented soymilk were 5.05% and 8.87%, respectively. When the concentration of the unfermented soymilk and that of the fermented soymilk increased to 200.00 mg/mL, the DPPH radical-scavenging ratio of the unfermented soymilk was 69.87%, and that of the fermented soymilk was 89.49%. The results are shown in FIG. 9. In comparison to the soymilk before fermentation, i.e. unfermented soy milk, the fermented soymilk at different concentrations significantly (P<0.05) or extremely significantly (P<0.01) increased the DPPH radical-scavenging ratios.

(2) Effect of the Reaction Time on DPPH Radical-Scavenging Ratio

8 mL of 0.1 mmol/L DPPH-ethanol solution was taken and mixed with 250 μL of the fermented soymilk followed by the addition of 1.75 mL of distilled water. The mixed solution was reacted in the dark at 25° C. after being shaken adequately. The mixed solution were taken out respectively at different reaction time periods, including 30 min, 2 h, 6 h, 24 h, 48 h, 72 h, 96 h, and 120 h, and the DPPH radical-scavenging ratio was calculated.

In the present disclosure, the DPPH free radical scavenging capacity of soymilk before and after fermentation was determined during different reaction time periods, and the results are shown in FIG. 10. The results of the study showed that the scavenging capacity increased significantly with time during the first 30 min to 24 h of the reaction time period; the DPPH radical-scavenging ratio of the unfermented soymilk increased from 13.63% to 34.30% when the reaction time period increased from30 min to 24 h; the DPPH radical-scavenging ratio of the fermented soymilk increased from 35.86% to 67.87% when the reaction time period increased from 30 min to 24 h. After 24 h of reaction, the DPPH radical-scavenging ratio of the unfermented soymilk basically did not increased with the prolonged reaction time. However, in case of the fermented soymilk, the DPPH radical-scavenging ratio still increased slowly with the prolonged reaction time. The DPPH radical-scavenging ratio of the fermented soymilk reached 81.61% at the reaction time period of 120 h.

Although embodiments of the present disclosure have been described in detail, it should be understood that various changes, substitutions and alterations can be made herein without departing from the spirit and scope of the disclosure as defined by the appended claims.

Moreover, the scope of the present disclosure is not intended to be limited to the particular embodiments described here. As one of ordinary skill in the art will readily appreciate from the disclosure of the present disclosure that processes, machines, manufacture, compositions of matter, means, methods, or steps, presently existing or later to be developed, may perform substantially the same function or achieve substantially the same result as the corresponding embodiments described herein. Accordingly, the appended claims are intended to include within their scope such processes, machines, manufacture, compositions of matter, means, methods, or steps.

What is claimed is:

1. A Lactobacillus acidipiscis HAU-FR₇, which was deposited in China General Microbiological Culture Collection Center (CGMCC) with a deposit number of CGMCC NO. 19253.

2. Use of the Lactobacillus acidipiscis HAU-FR₇ of claim 1 in fermentation of soymilk.

3. A fermented soymilk, which is prepared by using the Lactobacillus acidipiscis HAU-FR₇ of claim 1.

4. A preparation method of the fermented soymilk of claim ₃, comprising steps of:
inoculating a cultural broth of the Lactobacillus acidipiscis HAU-FR₇ in a logarithmic growth phase into a soymilk; and
fermenting the soymilk at 25° C.-42° C. for a number of hours to obtain the fermented soymilk, the number of hours in a range of ₃6 hours to ₅o hours.

5. The preparation method of the fermented soymilk of claim ₄, wherein an inoculation amount of the cultural broth of the Lactobacillus acidipiscis HAU-FR₇ is in a range of 8% to 12% of a volume of the soymilk.

6. The preparation method of the fermented soymilk of claim ₅, further comprising:
inoculating an activated bacterial solution of the Lactobacillus acidipiscis HAU-FR₇ into a De Man-Rogosa-Sharp (MRS) liquid medium at an inoculation amount of ₄%-6% (v/v) to obtain an inoculated solution; and
culturing the inoculated solution at ₃₅° C.-₄0° C. for a number of hours to obtain the culture broth of the Lactobacillus acidipiscis HAU-FR₇, the number of hours in a range of 20 hours to 25 hours.

7. The preparation method of the fermented soymilk of claim ₄, wherein the soymilk is prepared by mixing and pulverizing soybeans and water at a weight-to-volume ratio of 1:6, wherein a unit of weight is gram, and a unit of volume is milliliter.

8. The preparation method of the fermented soymilk of claim ₄, further comprising:
inoculating an activated bacterial solution of the Lactobacillus acidipiscis HAU-FR₇ into a MRS liquid medium at an inoculation amount of ₄%-6% (v/v) to obtain an inoculated solution; and
culturing the inoculated solution at ₃₅° C.-₄0° C. for a number of hours to obtain the culture broth of the Lactobacillus acidipiscis HAU-FR₇, the number of hours in a range of 20 hours to 25 hours.

9. Use of the fermented soymilk of claim ₃ in preparation of foods, which have functions including anti-oxidation, anti-aging, enhancing immunity, or lowering blood pressure or blood lipid.


SEQUENCE LISTING (5 sequences)

SEQ ID NO: 1 - DNA, 20 bp, Artificial Sequence
  Synthetic
agagtttgatcctggctcag

SEQ ID NO: 2 - DNA, 19 bp, Artificial Sequence
  Synthetic
ggttaccttgttacgactt

SEQ ID NO: 3 - DNA, 1480 bp, Lactobacillus acidipiscis
acgctggcggcgtgcctaatacatgcaagtcgaacgcaatctttgactaatgagtgcttgcactcagcggtcaaagtgcgagtggcgaacgggtgagtaacacgtgggcaatctgcccaaaagtgggggataacacttggaaacaggtgctaataccgcatcaaccggctgaccgcatggtcggccgggcaaagacggcgtcagctgtcgcttttggatgagcccgcggcgtattaactagttggtaaggtaacggcttaccaaggtgatgatacgtagccgaactgagaggttgatcggccacattgggactgagacacggcccaaactcctacgggaggcagcagtagggaatcttccacaatggacgcaagtctgatggagcaacgccgcgtgtatgaagaaggtcttcggatcgtaaaatactgttgtcagagaagaacacgtgatagagtaactgctatggcgctgacggtatctgaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagaagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtggggagcgaacaggattagataccctggtagtccacgctgtaaacgatgaatgctaggtgttggagggtttccgcccttcggtgccgcagctaacgcactaagcattccgcctggggagtacgatcgcaagattgaaactcaaaggaattgacgggggcccgcacaagcggtggagcatgtggtttaattcgaagcaacgcgaagaaccttaccaggtcttgacatcttttgaccatctgagagatcagaatctcccttcggggacaaaatgacaggtggtgcatggctgtcgtcagctcgtgtcgtgagatgttgggttaagtcccgcaacgagcgcaacccttattgtcagttgccagcattcagttgggcactctggcgagactgccggtgacaaaccggaggaaggtggggatgacgtcaagtcatcatgccccttatgacctgggctacacacgtgctacaatggacgatacaacgagtcgcgagaccgcgaggtttagctaatctctgaaagtcgttctcagttcggatcgtaggctgcaactcgcctacgtgaagtcggaatcgctagtaatcgcggatcagcatgccgcggtgaatacgttcccgggccttgtacacaccgcccgtcacaccatgagagtttgtaacacccaaagccggtgcggtaaccattttggagccagccgtctaaggtgggacagatgattgggg

SEQ ID NO: 4 - DNA, 1490 bp, Lactobacillus acidipiscis
  misc_feature (1019)..(1019): n is a, c, g, or t
gacgaacgctggcggcgtgcctaatacatgcaagtcgaacgcaatctttgaccaatgagtgcttgcactcagcggtcaaagtgcgagtggcgaacgggtgagtaacacgtgggcaatctgcccaaaagtgggggataacacttggaaacaggtgctaataccgcatcaaccggctgaccgcatggtcggccgggcaaagacggcgtcagctgtcgcttttggatgagcccgcggcgtattaactagttggtaaggtaacggcttaccaaggtgatgatacgtagccgaactgagaggttgatcggccacattgggactgagacacggcccaaactcctacgggaggcagcagtagggaatcttccacaatggacgcaagtctgatggagcaacgccgcgtgtatgaagaaggtcttcggatcgtaaaatactgttgtcagagaagaacacgtgatagagtaactgctatggcgctgacggtatctgaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagaagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtggggagcgaacaggattagataccctggtagtccacgctgtaaacgatgaatgctaggtgttggagggtttccgcccttcggtgccgcagctaacgcactaagcattccgcctggggagtacgatcgcaagattgaaactcaaaggaattgacgggggcccgcacaagcggtggagcatgtggtttaattcgaagcaacgcgaagaaccttaccaggtcttgacatcttttgaccatctgagagatcagaanttcccttcggggacaaaatgacaggtggtgcatggctgtcgtcagctcgtgtcgtgagatgttgggttaagtcccgcaacgagcgcaacccttattgtcagttgccagcattcagttgggcactctggcgagactgccggtgacaaaccggaggaaggtggggatgacgtcaagtcatcatgccccttatgacctgggctacacacgtgctacaatggacgatacaacgagtcgcgagaccgcgaggtttagctaatctctgaaagtcgttctcagttcggatcgtaggctgcaactcgcctacgtgaagtcggaatcgctagtaatcgcggatcagcatgccgcggtgaatacgttcccgggccttgtacacaccgcccgtcacaccatgagagtttgtaacacccaaagccggtgcggtaaccattttggagccagccgtctaaggtgggacagatgattggggtgaag

SEQ ID NO: 5 - DNA, 1489 bp, Lactobacillus pobuzihii
gacgaacgctggcggcgtgcctaatacatgcaagtcgaacgcaatcttttaacaatgagtgcttgcactcagcgttttaagtgcgagtggcgaacgggtgagtaacacgtgggcaatctgcccaaaagtgggggataacacttggaaacaggtgctaataccgcatcaaccgactgaccgcctggtcggtcgggcaaagacggcgtcagctgtcgcttttggatgagcccgcggcgtattaactagttggtaaggtaacggcttaccaaggtgatgatacgtagccgaactgagaggttgatcggccacattgggactgagacacggcccaaactcctacgggaggcagcagtagggaatcttccacaatggacgcaagtctgatggagcaacgccgcgtgtatgaagaaggtcttcggatcgtaaaatactgttgtcagagaagaacacgggataaagtaactattgttccgctgacggtatctgaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagaagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtggggagcgaacaggattagataccctggtagtccacgctgtaaacgatgaatgctaggtgttggagggtttccgcccttcggtgccgcagttaacgcactaagcattccgcctggggagtacgatcgcaagattgaaactcaaaggaattgacgggggcccgcacaagcggtggagcatgtggtttaattcgaagcaacgcgaagaaccttaccaggtcttgacatcttttgaccatctgagagatcagaatttcccttcggggacaaaatgacaggtggtgcatggctgtcgtcagctcgtgtcgtgagatgttgggttaagtcccacaacgagcgcaacccttattgtcagttgccagcattgagttgggcactctggcgagactgccggtgacaaaccggaggaaggtggggatgacgtcaagtcatcatgccccttatgacctgggctacacacgtgctacaatggacgatacaacgagtcgcgagaccgcgaggtttagctaatctctgaaagtcgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtgcggtaaccatttggagccagccgtctaaggtgggacagatgattggggtgaag